(19) United States
(12) Patent Application Publication
(10) Pub. No.: US 2016/0067295 A1
(43) Pub. Date: Mar. 10, 2016

(54) WALNUT EXTRACTS FOR NUTRACEUTICAL APPLICATIONS

(71) Applicant: OMNICA GMBH, HAMBURG (DE)

(72) Inventor: Thomas Eidenberger, Steyr (AT)

(21) Appl. No.: 14/942,223

(22) Filed: Nov. 16, 2015

Related U.S. Application Data
(62) Division of application No. 12/838,759, filed on Jul. 19, 2010.
(60) Provisional application No. 61/226,780, filed on Jul. 20, 2009.

(51) Int. Cl. A61K 36/52; A61K 31/683; A61K 31/688; A61K 31/661

(52) U.S. Cl. CPC A61K 36/52; A61K 31/661; A61K 31/683; A61K 31/688

(57) ABSTRACT

The invention describes to walnut oil extracts that have increased amounts of enriched in phospholipids, especially phosphatidylethanolamine (PE), phosphatidylcholine (PC), and phosphatidylinositol (PI) relative to walnut oil found in naturally occurring walnuts. It has been found that there are synergistic therapeutic effects when PE/PC and/or PI are combined.

### CROSS REFERENCE TO RELATED APPLICATIONS

This application is a divisional application of U.S. Ser. No. 12/838,759, filed Jul. 19, 2010, which claims benefit under 35 U.S.C. §119(e) to U.S. Provisional Patent Application Ser. No. 61/226,780, entitled “Walnut extracts for nutraceutical applications”, filed Jul. 20, 2009, the contents of which are incorporated herein by reference in their entirety.

### FIELD OF THE INVENTION

The invention relates generally to walnut kernel extracts that have increased amounts of enriched in phospholipids, especially phosphatidylethanolamine (PE), phosphatidylcholine (PC), phosphatidylserine (PS) and phosphatidylinositol (PI) relative to walnut oil found in naturally occurring walnuts. It has been found that there are synergistic therapeutic effects when PE/PC and/or PI are combined.

### BACKGROUND OF THE INVENTION

Walnut kernels generally contain about 60% oil but this can vary from 52 to 70% depending on the cultivar, location grown and irrigation rate. The major constituents of the oil are triglycerides, free fatty acids, diglycerides, monoglycerides, sterols and sterol esters. Polar constituents, such as phosphatides are only present in minor quantities. The major fatty acids found in walnut oil are oleic (18:1), linoleic (18:2) and linolenic (18:3) acids. The ratios of these to each other are important to the economic and nutritional value of the nut. A lower linoleic and linolenic acid content may give a longer shelf life, and monounsaturated fatty acids may be more desirable because of their potential health benefits.

There has been a general belief that walnuts, and walnut oil, have therapeutic benefits in terms of overall health and, in particular, mental health.

However, which beneficial component(s) of the walnut oil are not well understood.

Therefore, a need exists for a better understanding of the active agent(s) that overcomes one or more of the current disadvantages noted above.

### BRIEF SUMMARY OF THE INVENTION

The present invention provides walnut extracts that provide lipid compositions that are enriched in phospholipids, such as phosphatidylethanolamine (PE), phosphatidylcholine (PC), and phosphatidylinositol (PI) relative to walnut oil found in naturally occurring walnuts. The compositions can further include sphingolipids, such as sphingomyelin. It has been found that by utilizing specific ratios of PE to PC, surprising benefits for brain health can be achieved, including increased cognitive function and treatment of Alzheimer's disease.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 provides a graphical representation of the inhibitory effects of four phospholipid containing walnut extracts of the invention on fibrillization.

FIG. 2 provides a graphical representation of the inhibitory effects of two walnut phospholipid extracts of the invention on fibrillization in comparison to soy derived phospholipids.

FIG. 3 is a graphical representation comparing the defibrillization efficacy of soy derived phospholipids and four phospholipid containing walnut extracts of the invention.

FIG. 4 is a graphical representation of the inhibition of acetylcholinesterase by four phospholipid containing walnut extracts of the invention.

FIG. 5 is a graphical representation of the inhibition of acetylcholinesterase by soy derived phospholipids in comparison to two phospholipid containing walnut extracts of the invention.

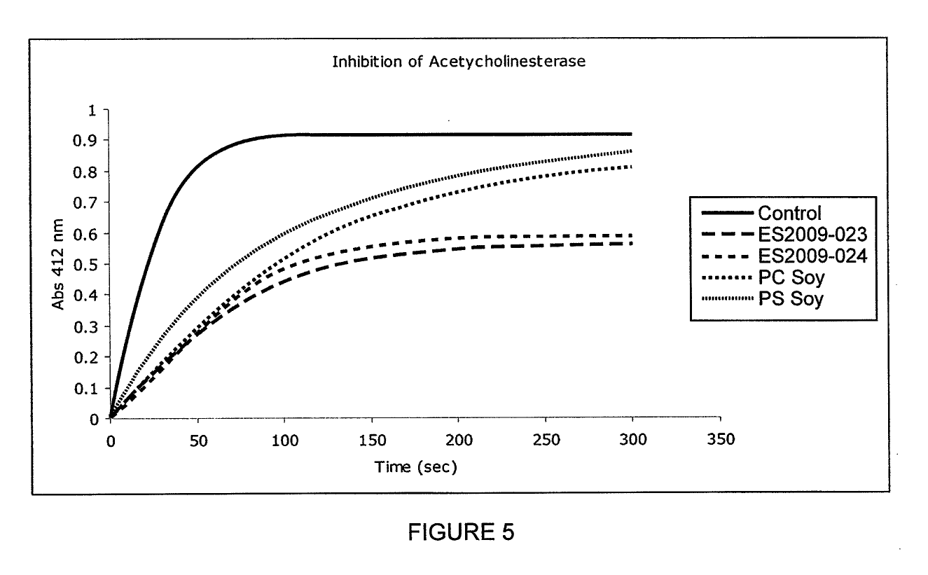

FIG. 6 is a mass spectrum of ES 2010-2007 in positive mode. The larger peak consists of phosphatidylcholine and sphingomyelins.

FIG. 7 is a mass spectrum of ES 2010-2008 in positive mode. The larger peak consists of phosphatidylcholine and sphingomyelins.

FIG. 8 is a mass spectrum of ES 2010-2007 in negative mode. The peaks are mainly PI and PE.

FIG. 9 is a mass spectrum of ES 2010-2008 in negative mode. The peaks are mainly PI and PE.

FIG. 10 is a mass spectrum of ES 2010-007.

FIG. 11 is a mass spectrum of ES 2010-008.

FIG. 12 is a mass spectrum of ES 2010-007.

FIG. 13 is a mass spectrum of ES 2010-008.

FIG. 14 is a mass spectrum of ES 2010-007, negative mode.

FIG. 15 is a mass spectrum of ES 2010-008, negative mode.

FIG. 16 depicts the inhibition of fibrillization by walnut extracts.

FIG. 17 depicts the percentage of defibrillization by walnut extracts.

FIG. 18 depicts the inhibition of acetylcholinesterase by walnut extracts of the invention.

### DETAILED DESCRIPTION

In the specification and in the claims, the terms “including” and “comprising” are open-ended terms and should be interpreted to mean “including, but not limited to. . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The present invention provides walnut extracts that provide lipid compositions that are enriched in phospholipids, such as phosphatidylethanolamine (PE), phosphatidylcholine (PC), and phosphatidylinositol (PI) relative to walnut oil found in naturally occurring walnuts.

It should be understood that the walnut extracts and compositions thereof described throughout include extracts that have been isolated, separated, or purified from walnuts. Therefore, the walnut extract or composition, is non-naturally occurring and has been acted upon to increase certain of the constituents within the walnut's physical/chemical make up. Certain of the naturally occurring constituents of the walnut physical/chemical make up may be removed or diminished by the isolation, separation or purification process. Thus the extract is enriched in desired components that provide useful advantages over naturally occurring walnut. Such desired components include, for example, PE, PC and PI.

The extracts and compositions of the invention differ from walnut oil. Walnut oil is generally cold pressed to simply crush the nut and collect the resultant oil and any constituents that remain suspended in the oil.

In one aspect, the phospholipid composition of the present invention has a ratio of greater than about 0.88 of PI/PC by weight, more particularly greater than about 1, even more particularly greater than about 1.5 and even greater than about 2.

In another aspect, the phospholipid composition of the present invention has a ratio of greater than about 0.40 of PI/PE by weight, more particularly greater than about 0.5, even more particularly greater than about 1 and even greater than about 1.5.

In still another aspect, the ratio of PC:PE:PI:PS is approximately 30:20:40:10 in a composition of the invention. Ratios of phosphatidyl-lipids/sphingosine-lipids is about 70/30 by weight.

In another aspect, the phospholipid composition of the present invention has a percentage of greater than about 80% of unsaturated fatty acids of the composition. For example, the unsaturated fatty acids can be, 14:1, 16:1, 18:1, 18:2, 18:3 and/or 22:1 unsaturated fatty acids. Saturated fatty acids can include 12:0, 14:0, 16:0, 18:0, 20:0 and/or 22:0 saturated fatty acids. Typically the range of fatty acids to unsaturated fatty acids is from about 15 to about 20%/80 to about 85% by weight, favoring the unsaturated fatty acids.

In one aspect, the total amount of phospholipids in the extracts of the invention is from about 80 to about 85% by weight.

In one embodiment, the present invention provides compositions that include one or more phospholipids and one or more sphingolipids, wherein the phospholipids include PE and PC. In such embodiments, the ratio of PE to PC is from about 1:1 to about 2:1 by weight and in particular from about 1.6:1 to about 1.7:1 by weight. The compositions can further include PI and the ratio of PE to PI is from about 2:1 to about 3:1 by weight and in particular from about 2.3:1 to about 2.7:1 by weight. In a particular embodiment, the ratio of PC:PI:PE is about 3:2:5 by weight. In an exemplary embodiment, the composition includes greater than 0.5% phospholipids by weight and in particular greater than 1.5% by weight. In particular, the composition can have a ratio of unsaturated fatty acid to saturated fatty acid from about 2:1 to about 2.5:1 by weight. The composition can further include greater than about 0.05% sphingolipid by weight.

In a particular embodiment the ratios for the components PC/PI/PE are from about 3:(2-4):(2-5). In yet another embodiment, the ratios for the components PC/PI/PE/PS are from about 3:(2-4):(2-5):(0.1-1).

In still another embodiment, ratios of about (20-50):30:(20-40) of PE:PC:PI are within the scope of the present invention.

The lipid compositions of the invention are beneficial to cognitive health. The inhibition of acetylcholinesterase (AChE) demonstrates that the lipid compositions are helpful to treat or improve Alzheimer's disease.

Furthermore, due to the multiple applications of the walnut extract (the phospholipid compositions), it is possible for walnut extract from the present invention to treat or improve heart disease, cancer, stroke, arteriosclerosis, etc.

The present invention further provides a combination of gingko extract and the lipid compositions as above mentioned. This combined product can improve the nutritional level of gingko extract, such as increased bioavailability.

Liposome formulation are widely used as media or as a carrier to transfer active ingredients. The unique walnut extract compositions of the invention can act as carriers, e.g., they can be formulated as liposome formulations, to deliver active agents such as drugs.

The compositions described herein can further include sphingolipids, such as sphingomyelin.

The compositions of the invention can be provided in various forms and incorporated into various products such as in food stuffs, nutraceuticals and with a drug.

The compositions of the invention can be incorporated into various foods, drinks, snacks, etc. In one aspect, the composition can be sprinkled onto a food product, prior to consumption. If sprinkled onto a food product, a suitable carrier such as starch, sucrose or lactose, can be used to help distribute the concentration of the compositions making it easier to apply to the food product.

The compositions of the present invention can also be provided as supplements in various prepared food products. For the purposes of this application, prepared food product means any natural, processed, diet or non-diet food product to which a composition of the invention has been added. The compositions of the present invention can be directly incorporated into many prepared diet food products, including, but not limited to diet drinks, diet bars and prepared frozen meals. Furthermore, the compositions of the inventions can be incorporated into many prepared non-diet products, including, but not limited to candy, snack products such as chips, prepared meat products, milk, cheese, yogurt, sport bars, sport drinks, mayonnaise, salad dressing, bread and any other fat or oil containing foods. As used herein, the term “food product” refers to any substance fit for human or animal consumption.

The compositions of the invention can be added to various drinks, such as fruit juices, milkshakes, milk, etc.

The preferred method of administration is oral. The compositions of the invention can be formulated with suitable carriers such as starch, sucrose or lactose in tablets, capsules, solutions, syrups and emulsions. The tablet or capsule of the present invention can be coated with an enteric coating that dissolves at a pH of about 6.0 to 7.0. A suitable enteric coating, which dissolves in the small intestine but not in the stomach, is cellulose acetate phthalate.

Formulation of the compositions of the invention into a soft gel capsule can be accomplished by many methods known in the art. Often the formulation will include an acceptable carrier, such as an oil, or other suspending or emulsifying agent.

Suitable optional carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, wheat germ oil, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The compositions of the invention can also include a nutraceutical. The term “nutraceutical” is recognized in the art and is intended to describe specific chemical compounds found in foods that can prevent disease or ameliorate an undesirable condition.

The compositions of the invention can further include various ingredients to help stabilize, or help promote the bioavailability of the components of the beneficial compositions of the invention or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals can be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Various additives can be incorporated into the present compositions. Optional additives of the present composition include, without limitation, hyaluronic acid, phospholipids, starches, sugars, fats, antioxidants, amino acids, proteins, flavorings, coloring agents, hydrolyzed starch(es) and derivatives thereof or combinations thereof.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, camosic acid and rosmanol found in rosemary and hawthorn extract, proanthocyanidins such as those found in grapeseed or pine bark extract, and green tea extract.

Compositions comprising the compositions of the invention can be manufactured by methods of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries that facilitate processing of the compositions into preparations that can be used.

The compositions of the invention can take a form suitable for virtually any mode of administration, including, for example, oral, buccal, systemic, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives.

Alternatively, the injectable formulation can be provided in powder form for reconstitution with a suitable vehicle, including but not limited to sterile pyrogen free water, buffer, dextrose solution, etc., before use. To this end, the compositions can be dried by any art-known technique, such as lyophilization, and reconstituted prior to use.

For transmucosal administration, penetrants appropriate to the barrier to be permeated are used in the formulation. Such penetrants are known in the art.

For oral administration, the compositions of the invention can take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

Preparations for oral administration can be suitably formulated to give controlled release of the composition as is well known.

For buccal administration, the compositions can take the form of tablets or lozenges formulated in conventional manner.

For rectal and vaginal routes of administration, the compositions can be formulated as solutions (for retention enemas) suppositories or ointments containing conventional suppository bases such as cocoa butter or other glycerides.

For nasal administration or administration by inhalation or insufflation, the compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflators (for example capsules and cartridges comprised of gelatin) can be formulated containing a powder mix of the compound and a suitable powder base such as lactose or starch.

For prolonged delivery, the compositions can be formulated as a depot preparation for administration by implantation or intramuscular injection. The compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the compositions for percutaneous absorption, can be used. To this end, permeation enhancers can be used to facilitate transdermal penetration of the compositions. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems can be employed. Liposomes and emulsions are well-known examples of delivery vehicles that can be used to deliver compositions of the invention. Certain organic solvents such as dimethylsulfoxide (DMSO) can also be employed, although usually at the cost of greater toxicity.

The compositions can, if desired, be presented in a pack or dispenser device, which can contain one or more unit dosage forms containing the compositions of the invention. The pack can, for example, comprise metal or plastic foil, such as a blister pack. The pack or dispenser device can be accompanied by instructions for administration.

The following paragraphs enumerated consecutively from 1 through 30 provide for various aspects of the present invention. In one embodiment, in a first paragraph (1), the present invention provides A walnut extract composition comprising phosphatidylethanolamine (PE) and phosphatidylcholine (PC), wherein the ratio of PE to PC is greater than about 0.43 by weight.

2. The walnut extract composition of paragraph 1, further comprising phosphatidylinositol (PI).

3. A walnut extract composition comprising phosphatidylinositol (PI) and phosphatidylcholine (PC), wherein the ratio of PI to PC is greater than about 0.88 by weight.

4. The walnut extract composition of paragraph 3, further comprising phosphatidylethanolamine (PE).

5. A walnut extract composition comprising phosphatidylinositol (PI) and phosphatidylethanolamine (PE), wherein the ratio of PI to PE is greater than about 0.4 by weight.

6. The walnut extract composition of paragraph 5, further comprising phosphatidylcholine (PC).

7. The walnut extract composition lipid composition of any of paragraphs 1 through 6, further comprising a sphingolipid.

8. The walnut extract of paragraph 7, wherein the sphingolipid is sphingomyelin.

9. The walnut extract of paragraph 7, wherein the ratio of phospholipids/sphingolipids is above 5:1 by weight.

10. The walnut extract according to any of the preceding paragraphs wherein the ratio of the unsaturated/saturated fatty acids is greater than about 1:1 by weight.

11. A lipid composition derived from walnut extract comprising at least about 10% by weight of phospholipids.

12. The lipid composition of paragraph 11, wherein the weight percentage of phospholipids of the extract is at least about 20% or greater.

13. The lipid composition of paragraph 12, wherein the phospholipids are (PE), phosphatidylcholine (PC) and phosphatidylinositol (PI).

14. The lipid composition of paragraph 13, wherein the ratio of PE to PC is greater than about 0.43 by weight.

15. The lipid composition of paragraph 13, wherein the ratio of PI to PC is greater than about 0.88 by weight.

16. The lipid composition of paragraph 13, wherein the ratio of PI to PE is greater than about 0.4 by weight.

17. The walnut extract of any of paragraphs 1 through 16, further comprising a gingko extract.

18. A composition comprising the walnut extract according to any of the preceding paragraphs in combination with a food product.

19. A composition comprising the walnut extract according to any of preceding paragraphs 1 through 17 in combination with a beverage.

20. A composition comprising the walnut extract according to any of preceding paragraphs 1 through 17 in combination with a nutraceutical.

21. A composition comprising the walnut extract according to any of preceding paragraphs 1 through 17 in combination with a drug.

22. A method to treat a disease or condition related to the aggregation of β-amyloid comprising the step of providing a walnut extract or composition of any of paragraphs 1 through 21.

23. A method to treat a disease or condition related Alzheimer's disease comprising the step of providing a walnut extract or composition of any of paragraphs 1 through 21.

24. A method to treat or prevent conditions associate with a stroke comprising the step of providing a walnut extract or composition of any of paragraphs 1 through 21.

25. A method to improve blood-brain barrier function comprising the step of providing a walnut extract or composition of any of paragraphs 1 through 21.

26. A method to improve blood-brain barrier transport of an active agent across the blood-brain barrier comprising the step of providing a walnut extract or composition of any of paragraphs 1 through 21.

27. A method to improve or maintain cognitive health comprising the step of providing a walnut extract or composition of any of paragraphs 1 through 21.

28. A method to provide neuro-protection comprising the step of providing a walnut extract or composition of any of paragraphs 1 through 21.

29. A method to facilitate neural development comprising the step of providing a walnut extract or composition of any of paragraphs 1 through 21.

30. A method to facilitate cerebral metabolism comprising the step of providing a walnut extract or composition of any of paragraphs 1 through 21.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Example 1

1000 g fresh walnut kernel (from Moldova) was crushed, and then was extracted at 45° C. in a Soxhlet extractor mixed with 3000 mL chloroform/ethanol (90:10 v/v) for 3 hours. The reaction product was filtered and the cake was extracted twice with the same extraction solvent, e.g., 2 to 10 times the volume for the extraction. The filtrate from three extractions was combined and vacuum dried at below 50° C. to remove solvent to yield lipids. The determination of the acid value of total lipids was performed. The total lipids were mixed with 1% of vitamin E to avoid oxidation.

The lipids were mixed with 2 L acetone, and stirred for 60 minutes at 10° C. followed by filtration. The resultant cake was washed with approximately 5 g of acetone that was chilled to 10° C. The residue was vacuum dried to yield a polar lipid solid. This sample was referred to as ES2008-021.

According to the above process, another five lipid samples were prepared. The difference was based on varying ratios of extraction solvents. All samples with various solvent ratios are listed as below.

| Sample No. | Ratio of extraction solvent (v/v) |
|---|---|
| ES2008-021 | chloroform/ethanol (90:10) |
| ES2008-022 | chloroform/ethanol (85:15) |
| ES2009-022 | chloroform/ethanol (80:20) |
| ES2009-023 | chloroform/ethanol (75:25) |
| ES2009-024 | chloroform/ethanol (65:35) |
| ES2009-025 | chloroform/ethanol (60:40) |

Absolute fat content (Determination acc. to Soxhlet)

| Sample ID | % lipids (m/m) |
|---|---|
| ES2008-021 | 51.2 |
| ES2008-022 | 86.7 |

-Distribution of Fatty Acids

| Fatty Acid | ES2008-021 | ES2008-022 |
|---|---|---|
| 12:0 | <0.1 | 0.54 |
| 14:0 | <0.1 | 0.37 |
| 14:1 | <0.1 | 0.00 |
| 16:0 | 10.7 | 11.3 |
| 16:1 | <0.1 | <0.1 |
| 18:0 | 4.32 | 3.43 |
| 18:1 | 19.0 | 21.8 |
| 18:2 | 56.6 | 56.1 |
| 18:3 | 9.15 | 6.28 |
| 20:0 | 0.21 | 0.23 |

The ratio of unsaturated:saturated=85:15% (˜6:1).

Phosphatidyllipids Gravimetrically:

| | |
|---|---|
| ES2008-021 | 3.5% of the total lipid fraction |
| ES2008-022 | 1.5% of the total lipid fraction |

The ratio of PCs:PIs:PEs=30%:20%:50%

Sphingolipids Gravimetrically:

| | |
|---|---|
| ES2008-021 | 5.2% of the total lipid fraction |
| ES2008-022 | 3.6% of the total lipid fraction |

Phospholipids by HPLC/ELSD—Mass Spectrometry

| | |
|---|---|
| ES2008-021 | 22.2% (m/m) phosphatidylcholine (PC) |
| | 13.9% (m/m) other phosphatidyllipids |
| | expressed as PC |
| ES2008-022 | 5.4% (m/m) phosphatidylcholine (PC) |
| | 15.2% (m/m) other phosphatidyllipids expressed as PC |

Content of Phosphatidylserine by HPLC/ELSD

| | |
|---|---|
| ES2008-021 | 1.3% (m/m) |
| ES2008-022 | 0.5% (m/m) |

Below is described a test for inhibition of amyloid β-aggregation by walnut extracts of the present invention. The four samples have similar ability to inhibit amyloid β-aggregation.

Sample Preparation:

The walnut extracts as well as PC and PS from soy bean were dissolved in ethanol and diluted with an assay buffer solution to achieve the final test assay concentration.

Sample incubation:

100 μg Amyloid [A] β1-40 (Sigma-Aldrich, A 1075) were dissolved in 25 μl Tris-buffer. The samples were incubated for 3 days at 37° C. with constant shaking:

Active samples: 10 μl Amyloid β solution+40 μl TRIS-buffer including inhibitors (preferably dissolved in TRIS-buffer)

Reagent blank samples: 50 μl TRIS-buffer including inhibitors (preferably dissolved in TRIS-buffer)

Control samples: 10 μl Amyloid β solution+40 μl TRIS-buffer.

For each active sample, a corresponding reagent blank sample was provided.

Test Assay:

After 3 days, each sample was mixed with 2 μM (636.8 μg) Thioflavin T and 950 μl glycine/NaOH buffer (50 mM glycine, pH=9.0).

Fluorescence was measured at ex 435 nm/em 485 nm.

Data Evaluation:

Subtract fluorescence intensity for the corresponding “reagent blank samples” from each “active sample”. The resulting fluorescence intensity was compared with the fluorescence intensity measured for the control sample. % inhibition of aggregation was calculated by expressing the decrease in fluorescence intensity as % from the control.

The amyloid β aggregation, indicative for plagues seen in Alzheimer patients, was initiated in an in-vitro test assay system Inhibition of amyloid β aggregation was induced by incubation with walnut extracts or pure PS and PC from soy bean.

All walnut extracts yielded a similar inhibition characteristic when the sample concentration was adjusted to contain equal amounts of PC.

Following dilutions of the four samples were chosen on basis of the content of PC determined by HPLC/MS to obtain a stock solution of 15 mg PC/ml. Further dilutions down to 1 mg PC/ml were prepared using test assay buffer.

| | | |
|---|---|---|
| ES2009-022 | 458.7 μl | to 10 ml |
| ES2009-023 | 699.3 μl | to 10 ml |
| ES2009-024 | 316.05 μl | to 10 ml |
| ES2009-025 | 485.4 μl | to 10 ml |

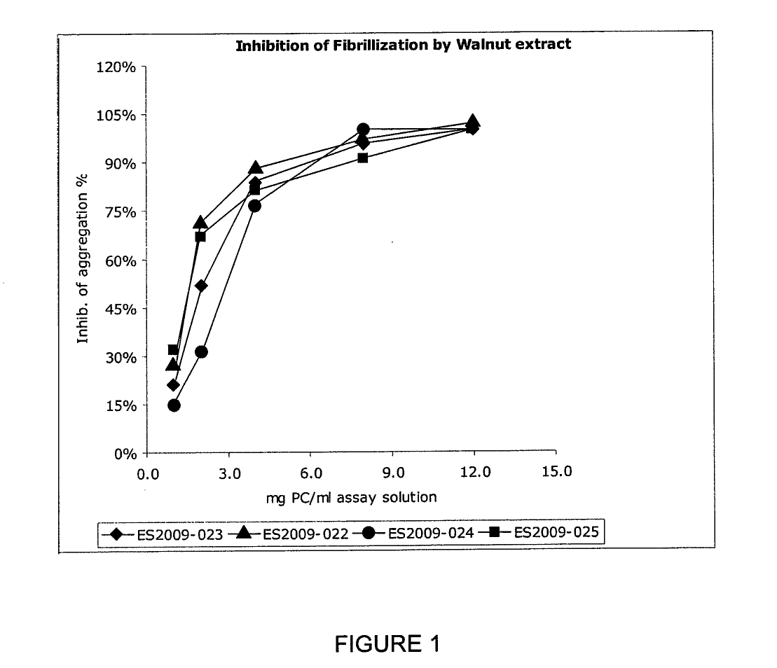

FIG. 1 demonstrates the efficiency of inhibition of fibrillization by the Walnut extracts of the invention based on the concentration of PC of the extract.

### Example 3

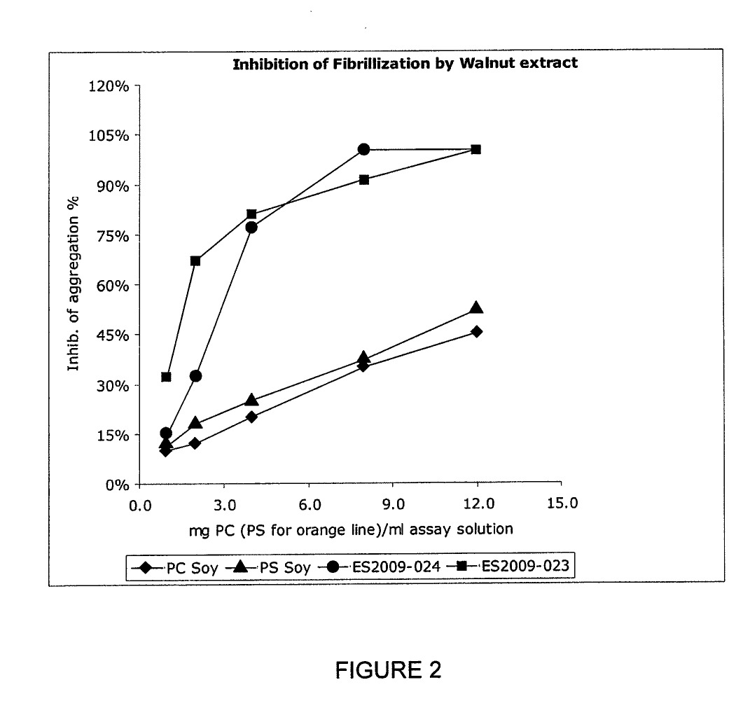

FIG. 2 provides data for the inhibition of amyloid aggregation by PC and PS from soy bean (pure compounds diluted to the concentrations given on the x-axis).

As can be noted from FIG. 2, the test results show that walnut extract samples are substantially more potent in the inhibition of amyloid aggregation than PS or PC from soy bean under comparable conditions.

### Example 4

In this example, the highest concentrations of the walnut extracts (12 mg PC/ml test assay for the walnut extracts, 12 mg PC Soy or PS Soy/ml) were evaluated for their ability to reverse an already formed aggregate of amyloid-β.

Defibrillization efficacy is indicative for treatment of Alzheimer disease.

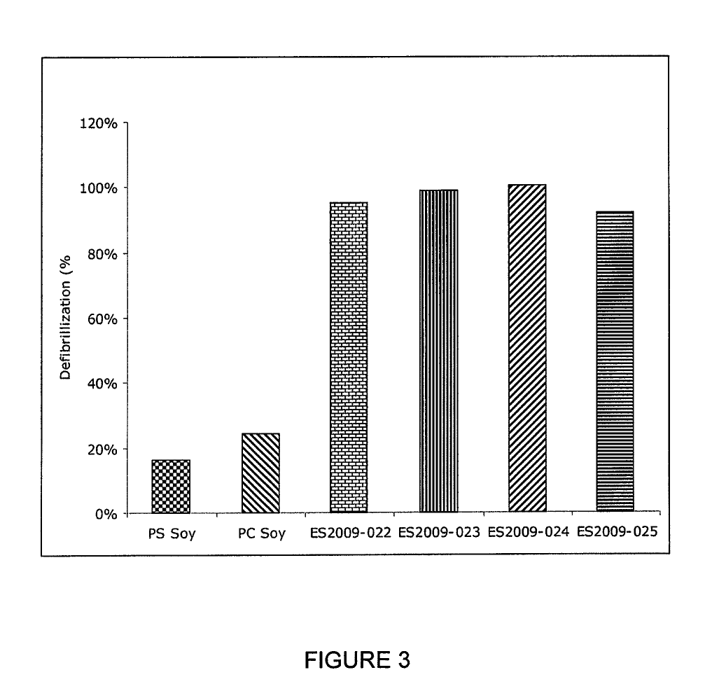

As seen in FIG. 3, PS or PC from Soy are less potent in defibrillization when compared to walnut extract samples.

### Example 5

This example is about general composition make up of the walnut extract samples:

| | | ±s.d. | n |
|---|---|---|---|
| | Nitrogen (%) | | |
| ES2009-022 | 1.265 | 0.030 | 3 |
| ES2009-023 | 3.237 | 0.015 | 3 |
| ES2009-024 | 1.284 | 0.039 | 3 |
| ES2009-025 | 4.533 | 0.033 | 3 |
| | Residue on ignition (%) | | |
| ES2009-022 | 9.13 | 0.125 | 3 |
| ES2009-023 | 8.35 | 0.093 | 3 |
| ES2009-024 | 11.0 | 0.147 | 3 |
| ES2009-025 | 9.16 | 0.114 | 3 |
| | Content HPLC/ELSD (%)* | | |
| ES2009-022 | 65.4 | | |
| ES2009-023 | 42.9 | | |
| ES2009-024 | 94.9 | | |
| ES2009-025 | 61.8 | | |
| | Phosphorous (%)** | | |
| ES2009-022 | 0.249 | 0.024 | 3 |
| ES2009-023 | 0.194 | 0.015 | 3 |
| ES2009-024 | 0.305 | 0.019 | 3 |
| ES2009-025 | 0.225 | 0.027 | 3 |
| | Dry Mass (%)*** | | |
| ES2009-022 | 98.2 | | |
| ES2009-023 | 99.1 | | |
| ES2009-024 | 97.2 | | |
| ES2009-025 | 96.5 | | |

* . . . as phosphatidylcholine
** . . . determined colorimetric from residue on ignition (“inorganic phosphorous”)
*** . . . 2 hours at 105° C.

Discussion of Results:

Dry mass, residue on ignition and content determined by HPLC/ELSD is consistent.

The content of nitrogen, indicative for N-containing phospholipids varies between the samples. That could mean the content of N-containing phospholipids is different or that in samples ES2009-023 and ES2009-025 some protein was co-extracted (which seems more likely).

### Example 6

%-Distribution of Fatty Acids

| Fatty Acid | ES2008-022 | ES2008-024 |
|---|---|---|
| 12:0 | <0.1 | 0.54 |
| 14:0 | <0.1 | 0.37 |
| 14:1 | <0.1 | 0 |
| 16:0 | 11.3 | 12.1 |
| 16:1 | <0.1 | <0.1 |
| 18:0 | 3.76 | 2.58 |
| 18:1 | 17.3 | 20.3 |
| 18:2 | 57.2 | 55.83 |
| 18:3 | 10.32 | 7.72 |
| 20:0 | 0.11 | 0.46 |
| unsaturated | about 85.02 | about 83.95 |
| saturated | about 15.37 | about 16.05 |

HPLC Conditions

A 150×3.2 mm Prevail silica column with 3 μm particle diameter (Alltech Associates, Inc.) was used. A pre-column with the same packing and internal diameter was used. The elution program was a linear gradient with 87.5:12:0.5 (v/v/v) chloroform:methanol:triethylamine buffer (pH 3, 1 M formic acid) at t=−0 minutes to 28:60:12 (v/v/v) at t=20 minutes. The mobile phase was brought back to the initial conditions at t=21 minutes and the column was allowed to equilibrate until the next injection at t=30 minutes. The flow was maintained at 0.5 mL/min, which resulted in a backpressure of 55 to 90 bar. The injection volume was 25 μL. The samples and the column were equilibrated at 40° C.

Detection:

ELSD (N₂ 1.5 L/min, nebulizing temperature 85° C.) for quantitative analysis of PL classes.

ESI-MS (positive mode, m/z 100-1200, GAIN 2, dwell time 100 msec, Fragmentor variable, Drying Gas 12 L N₂/min, Nebulizer pressure 50 psi, Temperature was 300° C., capillary voltage 3500 V) for qualitative analysis of PL-structures and confirmation of PL-classes.

Sample Preparation:

Samples were dissolved/suspended in chloroform/methanol=2:1 (v/v). Samples were diluted to fall into the calibration range (1-10 mg sample/ml solvent).

Calibration:

Available reference compounds (Sigma-Aldrich) were dissolved in chloroform/ methanol=2:1 (v/v) at concentrations of 20-200 μg/ml. In ELSD the response of the main PL classes is rather similar, so in case of lacking reference compounds, L- phosphatidyl-choline (P3841) serves as external reference standard. In that case results are expressed as “phospholipids as phosphatidyl-choline”. L- phosphatidyl-choline (P3841) is almost exclusively composed of 2 compounds eluting at the same time, so it is considered the most reliable calibration procedure.

Linearity can be obtained also from other reference compounds, but most the other standards split at least into 2 peaks when being injected onto the HPLC-system.

P3841-25MG : Sigma L-α-Phosphatidylcholine ˜99%, from bovine brain, lyophilized powder

| | ES2009-022 | ES2009-023 | ES2009-024 | ES2009-025 |
|---|---|---|---|---|
| Total phospholipds (g/sample) | 65.4 | 42.9 | 94.9 | 61.8 |
| PE (g/100 g Phospholipds) | 25.3 | 23.4 | 32.5 | 15.0 |
| PI (g/100 g Phospholipds) | 10.2 | 28.9 | 26.5 | 30.3 |
| PS (g/100 g Phospholipds) | 2.6 | 2.6 | 2.5 | 2.5 |
| PC (g/100 g Phospholipds) | 5.2 | 17.6 | 23.8 | 34.5 |
| SM (g/100 g Phospholipds) | 7.6 | 5.7 | 3.1 | 5.0 |
| Total (g/100 g Phospholipds) | 50.85 | 78.19 | 88.50 | 87.29 |
| PE (g/100 g sample) | 32.5 | 12.8 | 34.8 | 10.6 |
| PI (g/100 g sample) | 13.1 | 15.9 | 28.5 | 21.5 |
| PS (g/100 g sample) | 3.3 | 1.4 | 2.7 | 1.7 |
| PC (g/100 g sample) | 6.7 | 9.6 | 25.6 | 24.5 |
| SM (g/100 g sample) | 9.7 | 3.2 | 3.4 | 3.5 |

Sphingomyelins are between 2-10% of all phospholipids.

PS is between 1-10% of total phospholipids.

### Example 7

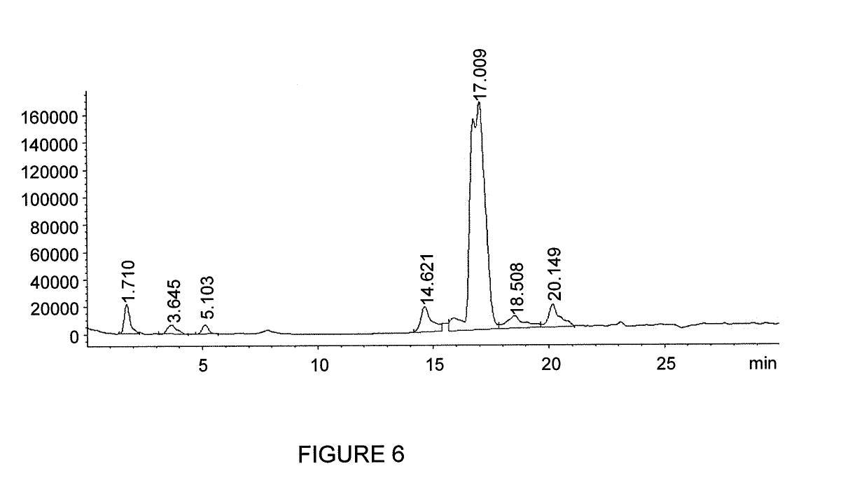

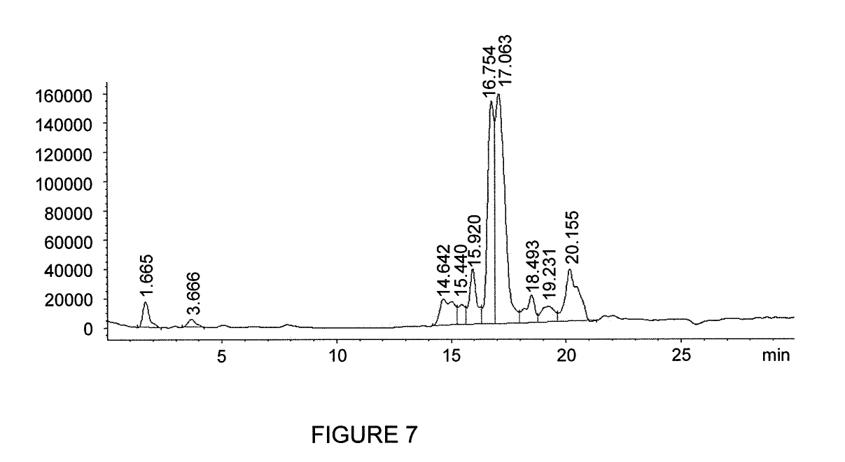

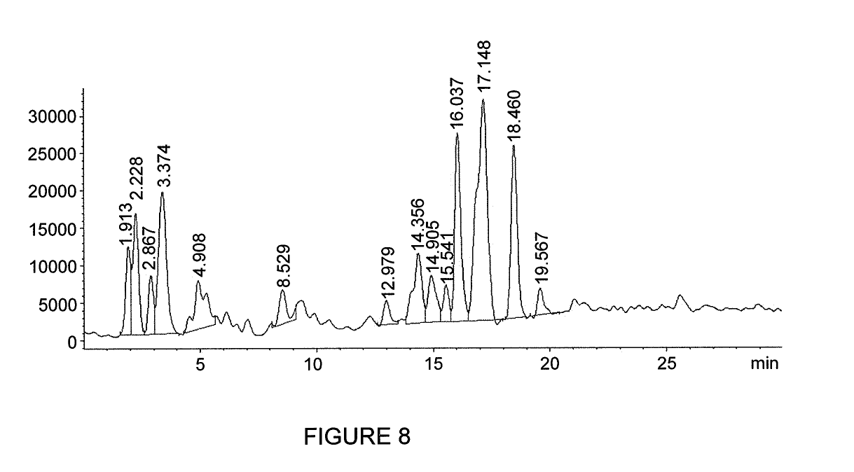

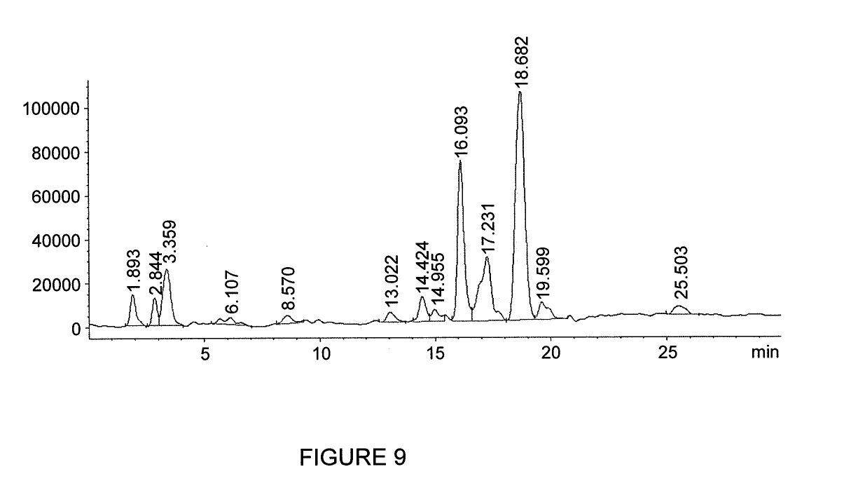

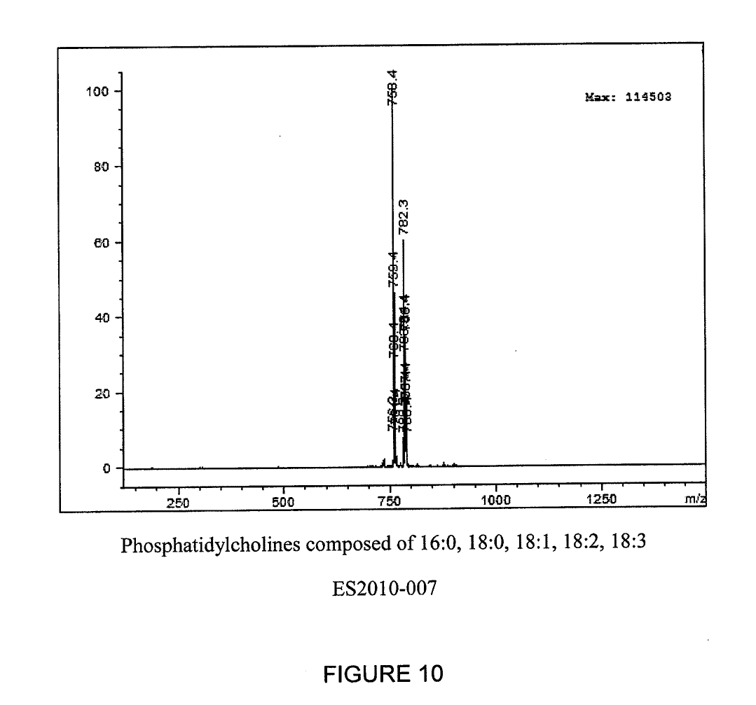

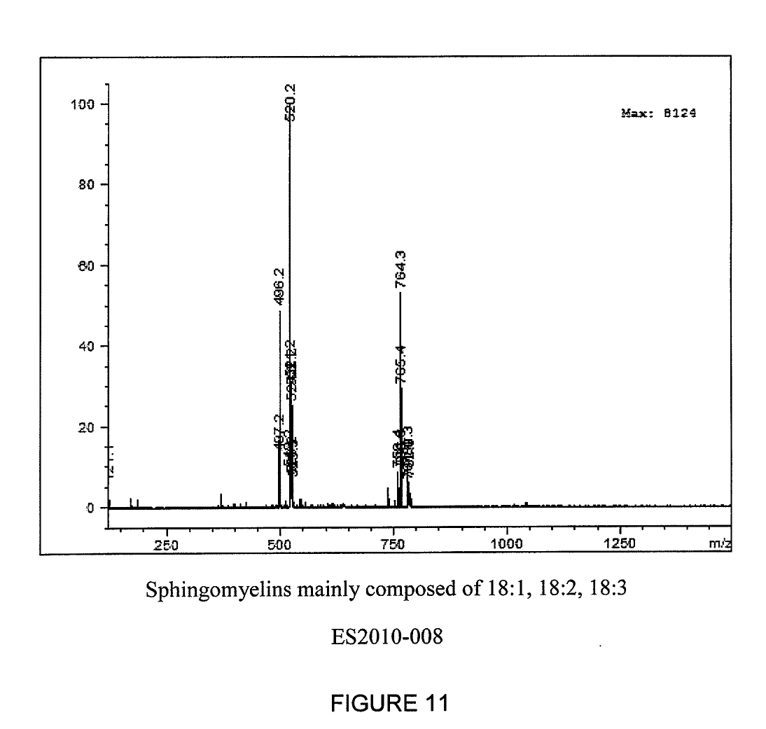

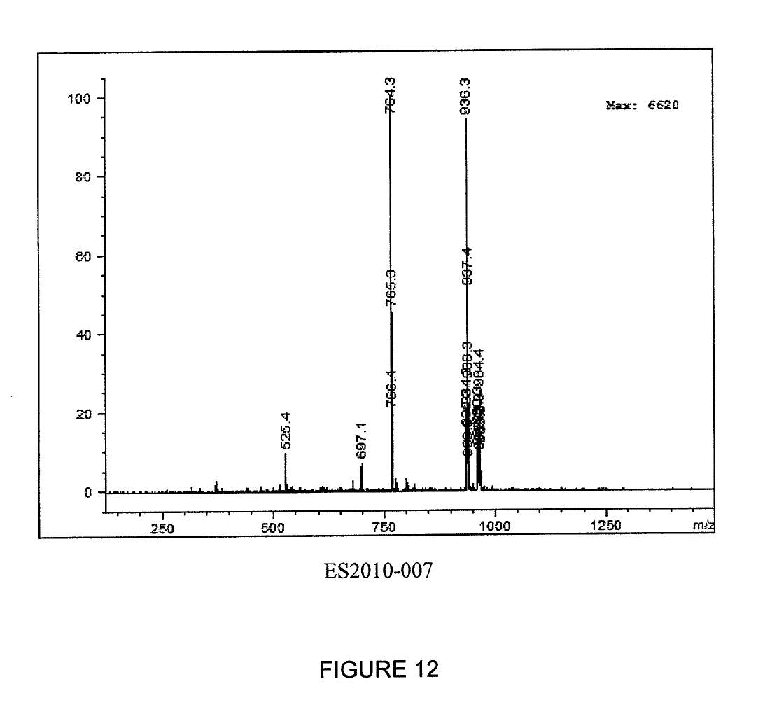

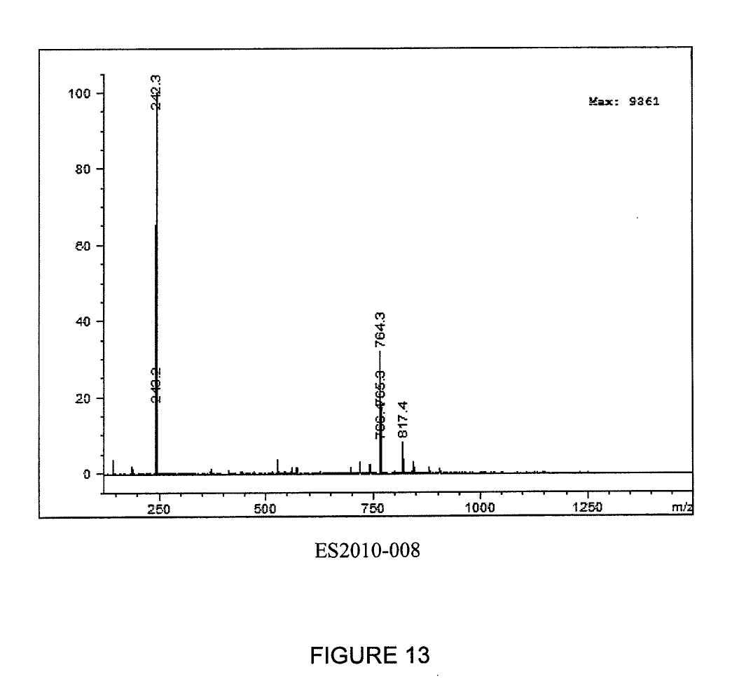

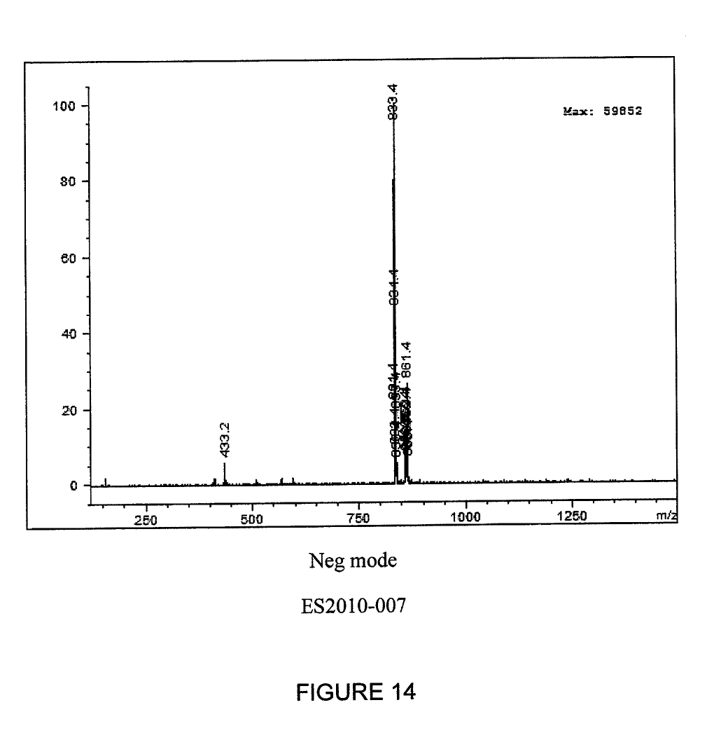

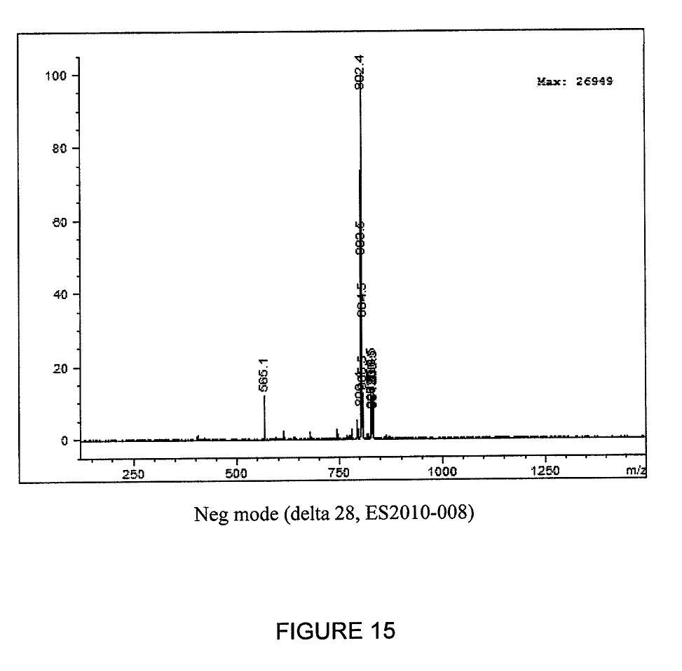

Samples ES2010-007 and ES2010-008 were prepared as above for samples ES2008-021 and ES2009-025 respectively. See FIGS. 6 through 15.

| Sample No. | | ±s.d. | n |
|---|---|---|---|
| | Residue on ignition (%) | | |
| ES2010-007 | 10.18 | 0.231 | 2 |
| ES2010-008 | 9.75 | 0.178 | 2 |
| | Content HPLC/ELSD (%)* | | |
| ES2010-007 | 32.5 | | |
| ES2010-008 | 27.4 | | |
| | Total Lipids (Soxhlet) | | |
| ES2010-007 | 39.6 | | |
| ES2010-008 | 32.4 | | |

* . . . as phosphatidylcholine

See FIG. 6 through 9.

%-Distribution of Fatty Acids

| Fatty Acid | ES2010-007 | ES2010-008 |
|---|---|---|
| 12:0 | <0.1 | <0.5 |
| 14:0 | <0.1 | <0.5 |
| 14:1 | <0.1 | <0.1 |
| 16:0 | 14.1 | 11.9 |
| 16:1 | <0.1 | <0.1 |
| 18:0 | 4.72 | 3.23 |
| 18:1 | 14.36 | 15.66 |
| 18:2 | 61.01 | 55.91 |
| 18:3 | 5.81 | 13.3 |
| 20:0 | <0.5 | <0.5 |

### Example 7

This example demonstrates the inhibition of human blood (acetyl) cholinesterase (Ellman method: use of 5,5′-dithiobis-2-nitrobenzoic acid (DTNB, Ellman's reagent) as a chromogen and record the level of cholinesterase activity as an increase of absorbance at 412 nm) by walnut extract samples.

Spectroscopic evaluation of acetyl cholinesterase activity was determined in the presence/absence of potentially inhibitory compounds. The test provides a coupling of choline liberated during the enzyme assay with Ellman's reagent to produce a coloured compound. The increase in absorption at 412 nm is measured over time to estimate the activity of the enzyme (i.e. the higher the absorption and the steeper, the more active is the enzyme).

Samples were dissolved in ethanol. Dilutions to achieve the final test concentration were prepared with ethanol/aqueous buffer=1:1 (v/v). During incubation with the enzyme the absorption was measured continuously at 412 nm.

The concentration of the samples was adjusted to 1 mg Phosphatidylcholine as determined by HPLC/MS per ml test solution. The following dilutions were prepared in the same way:

| | | |
|---|---|---|
| ES2009-022 | 30.58 μl | to 10 ml |
| ES2009-023 | 46.62 μl | to 10 ml |
| ES2009-024 | 21.07 μl | to 10 ml |
| ES2009-025 | 32.36 μl | to 10 ml |

This was confirmed with MS analysis:

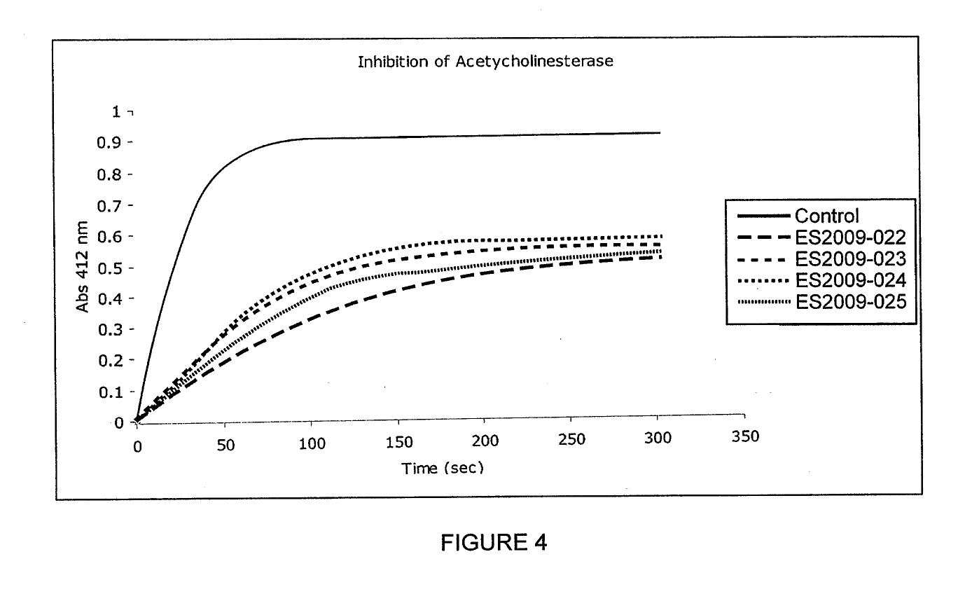

As seen in FIG. 4, the inhibition achieved by walnut extract samples is almost equivalent for all samples tested.

### Example 8

This example provides comparisons of the walnut extract (E2009-023 and ES2009-024) with phosphatidylcholine (PC) and phosphatidylserine (PS) from soy bean.

The concentration for the 2 samples was 1 mg/ml test solution.

The table provided below shows the remaining activities within 5 minutes of incubation.

| Sample | % activity of AchE | % inhibited part of activity of AchE |
|---|---|---|
| Control | 100.0 | 0 |
| PS Soy Bean | 94.0 | 6.0 |
| PC Soy Bean | 88.2 | 11.8 |
| ES2009-023 | 64.1 | 35.9 |
| ES2009-024 | 61.5 | 38.5 |

Therefore, it can be seen that PS and PC derived from soybean were almost inactive in inhibiting acetyl cholinesterase. Under the equivalent concentrations, the PC in walnut extract samples showed a significant inhibitory effect over the soybean PC.

### Example 9

Inhibition of amyloid Aβ-aggregation by ES2008-021 and ES2008-022 -Fibrillization assay

The amyloid β aggregation, indicative for plagues seen in Alzheimer patients, was initiated in an in-vitro test assay system.

Inhibition of fibrillization was induced by incubation with walnut extract. At each concentration of walnut extract (0.625, 1.25, 2.5, 5.0 or 7.5 μl ES2008-022 or 0.625, 1.25, 2.5, 5.0 or 7.5 μl from a stock solution containing 125 mg ES2008-021/ml) three samples were incubated and evaluated.

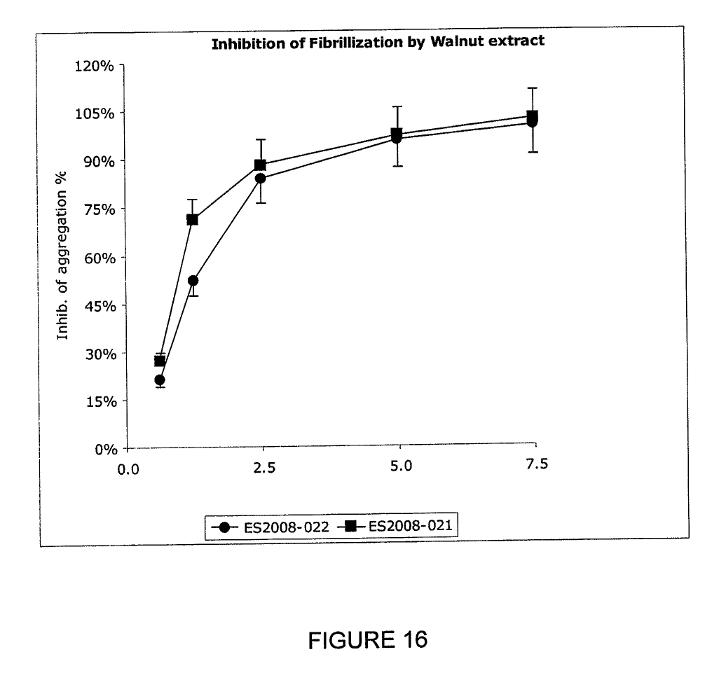

As seen in FIG. 16, 2.5 μl ES2008-022 (liquid form) or 2.5 μl ES2008-021 solution (representing 312 μg test article) are able to inhibit aggregation by approximately 90%.

Experimental—Fibrillization Assay

Walnut Extract:

The samples obtained were introduced directly after filtration (syringe filter 0.45 μm) into the test (ES2008-022) or after solubilization (125 mg ES2008-021/ml). the extract was used for the following experiments.

Sample Incubation:

Dissolve 100 μg Amyloid [A]β₁₋₄₀ (Sigma-Aldrich, A 1075) in 25 μl Tris-buffer

Incubate following samples for 3 days at 37° C. while constantly shaking:

Active samples: 10 μl Amyloid β solution+40 μl TRIS-buffer including inhibitors (preferably dissolved in TRIS-buffer)

Reagent Blank Samples:

50 μl TRIS-buffer including inhibitors (preferably dissolved in TRIS-buffer)

Control samples: 10 μl Amyloid β solution+40 μl TRIS-buffer

The above described samples were introduced at volumes of 2.5, 5.0 and 7.5 μl. For each active sample a corresponding reagent blank sample was introduced.

Test Assay:

After 3 days each sample is mixed with 2 μM (636.8 μg) Thioflavin T and 950 μl glycine/NaOH buffer (50 mM glycine, pH=9.0). Measure fluorescence at ex 435 nm/em 485 nm.

Data Evaluation:

Subtract fluorescence intensity for the corresponding “reagent blank samples ” from each “active sample”. Compare the resulting fluorescence intensity with the fluorescence intensity measured for the control sample. % inhibition of aggregation was calculated by expressing the decrease in fluorescence intensity as % from the control.

Dissolution of amyloid Aβ-aggregates by ES2008-021 and ES2008-022-Defibrillization assay

In a further extension of the test system, the above samples were evaluated for their ability to reverse an already formed aggregate of amyloid-β. Defibrillization efficacy is indicative for curing of Alzheimer disease.

Dissolution of amyloid Aβ-aggregates was induced by incubation with walnut extract. At each concentration of walnut extract (0.625, 1.25, 2.5, 5.0 or 7.5 μl ES2008-022 or 0.625, 1.25, 2.5, 5.0 or 7.5 μl from a stock solution containing 125 mg ES2008-021/ml) three samples were incubated and evaluated.

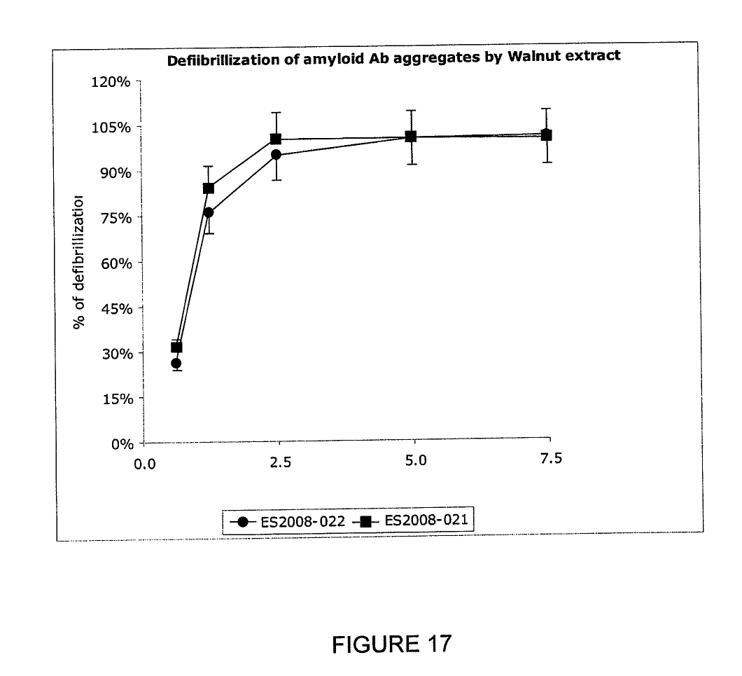

As seen in FIG. 17, 1.25 μl ES2008-022 (liquid form) or 1.25 μl ES2008-021 solution (representing 156 μg test article) are able to dissolve aggregates by almost 90%.

Experimental—Defibrillization Assay

Walnut Extracts:

The samples obtained were introduced directly after filtration (syringe filter 0.45 μm) into the test (ES2008-022) or after solubilization (125 mg ES2008-021/ml). The extract was used for the following experiments.

Sample Incubation:

Dissolve 100 μg Amyloid [A]β₁₋₄₀ (Sigma-Aldrich, A 1075) in 25 μl Tris-buffer

Keep sample at 37° C. for 24 hours while constantly shaking to produce aggregates.

Incubate following samples for 3 days at 37° C. while constantly shaking:

Active samples: 10 μl Amyloid β solution+40 μl TRIS-buffer including inhibitors (preferably dissolved in TRIS-buffer)

Reagent Blank Samples:

50 μl TRIS-buffer including inhibitors (preferably dissolved in TRIS-buffer)

Control samples: 10 μl Amyloid β solution+40 μl TRIS-buffer

The above described were introduced at volumes of 2.5, 5.0 and 7.5 μl. For each active sample a corresponding reagent blank sample was introduced.

Test Assay:

After 3 days each sample is mixed with 2 μM (636.8 μg) Thioflavin T and 950 μl glycine/NaOH buffer (50 mM glycine, pH=9.0). Fluorescence was measured at ex 435 nm/em 485 nm.

Data Evaluation:

Subtract fluorescence intensity for the corresponding “reagent blank samples” from each “active sample”. Compare the resulting fluorescence intensity with the fluorescence intensity measured for the control sample.

% dissolution of aggregation (defibrillization) was calculated by expressing the decrease in fluorescence intensity as % from the control.

Inhibition of human blood (acetyl)cholinesterase by ES2008-021 and ES2008-022-Ellman assay

Spectroscopic evaluation of acetylcholinesterase activity in presence/absence of potentially inhibiting compounds. Test principle is the coupling of choline liberated during the enzyme assay with Ellman's reagent.

ES2008-021 and ES2008-022 were diluted/dissolved with methanol. The concentrations were 10 mg sample/ml methanol. The amount added to the incubation assay was 30 μl for both samples.

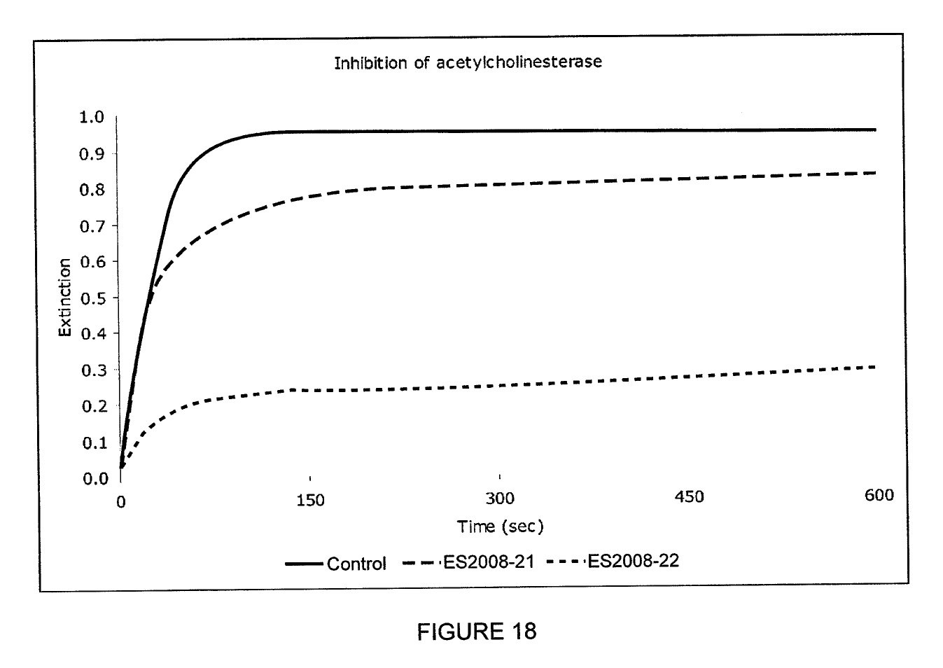

The increase in absorption at 412 nm was measured over time. As seen in FIG. 18, the inhibition achieved by ES2008-021 and ES2008-022 was different.

At almost equal concentrations, ES2008-021 (representing 300 μg test article) inhibited human acetylcholine-esterase by 70% whereas ES2008-022 inhibited by only 13%.

Experimental—Cholinesterase Assays

Inhibitor Solution:

Weigh in 10 mg of the corresponding sample (ES2008-021, ES2008-22) and add 1 ml methanol.

Chromogen Solution:

Mix 0.1 M Sodium phosphate buffer (pH=8, NaOH) with solution A at a ratio of 30:1.

Solution A:

Weigh in 39.6 mg 5,5′-dithio-bis[2-nitrobenzoic acid] and 15.0 mg NaHCO₃ into a 10 ml flask and fill up to the mark with 0.1 M Sodium-phosphate buffer (pH=7, NaOH).

Enzyme Solution:

Prepare a solution of human acetylcholinesterase containing 70 IU enzyme activity/ml H₂O.

Substrate solution: Weigh in 115.7 mg acetylthiocholine-iodide into a 10 ml flask and fill up to the mark with H₂O.

Mix 10 μl inhibitor solution with 185 μl chromogen solution and 10 μl enzyme solution. Incubate for 10 minutes at room temperature while shaking. Add 5 μl substrate solution and record the increase in absorption at 412 nm until no more increase is observed (3-5 minutes).

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method to treat a disease or condition related to the aggregation of β-amyloid, Alzheimer's disease, stroke, to improve blood-brain barrier function, to improve blood-brain barrier transport of an active agent across the blood-brain barrier, to improve or maintain cognitive health, to provide neuro-protection, to facilitate neural development and/or to facilitate cerebral metabolism comprising the step of providing to an individual an effective amount of a walnut extract composition comprising phosphatidylethanolamine (PE) and phosphatidylcholine (PC), wherein the ratio of PE to PC is greater than about 0.43 by weight, such that one or more of the conditions are treated, improved or facilitated.

2. The method of claim 1, further comprising phosphatidylinositol (PI).

3. The method of claim 2, further comprising a sphingolipid.

4. The method of claim 3, wherein the sphingolipid is sphingomyelin.

5. The method of claim 3, wherein the ratio of phospholipids/sphingolipids is above 5:1 by weight.

6. The method of claim 1, wherein the ratio of the unsaturated/saturated fatty acids is greater than about 1:1 by weight.

7. A method to treat a disease or condition related to the aggregation of β-amyloid, Alzheimer's disease, stroke, to improve blood-brain barrier function, to improve blood-brain barrier transport of an active agent across the blood-brain barrier, to improve or maintain cognitive health, to provide neuro-protection, to facilitate neural development and/or to facilitate cerebral metabolism comprising the step of providing to an individual an effective amount of a walnut extract composition comprising phosphatidylinositol (PI) and phosphatidylcholine (PC), wherein the ratio of PI to PC is greater than about 0.88 by weight, such that one or more of the conditions are treated, improved or facilitated.

8. The method of claim 7, further comprising phosphatidylethanolamine (PE).

9. The method of claim 8, further comprising a sphingolipid.

10. The method of claim 9, wherein the sphingolipid is sphingomyelin.

11. The method of claim 9, wherein the ratio of phospholipids/sphingolipids is above 5:1 by weight.

12. The method of claim 7, wherein the ratio of the unsaturated/saturated fatty acids is greater than about 1:1 by weight.

13. A method to treat a disease or condition related to the aggregation of β-amyloid, Alzheimer's disease, stroke, to improve blood-brain barrier function, to improve blood-brain barrier transport of an active agent across the blood-brain barrier, to improve or maintain cognitive health, to provide neuro-protection, to facilitate neural development and/or to facilitate cerebral metabolism comprising the step of providing to an individual an effective amount of a walnut extract composition comprising phosphatidylinositol (PI) and phosphatidylethanolamine (PE), wherein the ratio of PI to PE is greater than about 0.4 by weight, such that one or more of the conditions are treated, improved or facilitated.

14. The method of claim 13, further comprising phosphatidylcholine (PC).

15. The method of claim 14, further comprising a sphingolipid.

16. The method of claim 15, wherein the sphingolipid is sphingomyelin.

17. The method of claim 15, wherein the ratio of phospholipids/sphingolipids is above 5:1 by weight.

18. The method of claim 13, wherein the ratio of the unsaturated/saturated fatty acids is greater than about 1:1 by weight.
